(19) United States
(12) Patent Application Publication
(10) Pub. No.: US 2009/0137617 A1
(43) Pub. Date: May 28, 2009

(54) USE OF HAPTOGLOBIN GENOTYPING IN DIAGNOSIS AND TREATMENT OF CARDIOVASCULAR DISEASE

(76) Inventor: Andrew Levy, Haifa (IL)

Correspondence Address: Pearl Cohen Zedek Latzer, LLP, 1500 Broadway, 12th Floor, New York, NY, 10036, US

(21) Appl. No.: 12/276,963

(22) Filed: Nov. 24, 2008

Related U.S. Application Data
(60) Provisional application No. 60/996,552, filed on Nov. 23, 2007.

(51) Int. Cl. A61K 31/505; C12Q 1/68; A61K 31/355; A61K 31/435; A61K 31/35; A61K 31/40

(52) U.S. Cl. 514/275; 435/6; 514/458; 514/460; 514/423; 514/277

(57) ABSTRACT

This invention is directed to methods and compositions for the treatment of cardiovascular disorders. Specifically, the invention is directed to compositions comprising vitamin E, statins and/or glutathione peroxidase mimetics; methods of treating diabetic patients expressing the Hp-2-2 haptoglobin genotype; a method of inhibiting or suppressing a cardiovascular disorder in a diabetic subject, treating cardiovascular disease in subjects exhibiting the Haptoglobin Hp-2-2 genotype; and methods of treating cardiovascular disease in subjects exhibiting the Haptoglobin Hp-2-2 genotype.

### CROSS-REFERENCE TO RELATED APPLICATION

This application claims priority to U.S. provisional patent application Ser. No. 60/996,552, filed Nov. 23, 2007, which is incorporated herein by reference in its entirety.

### FIELD OF INVENTION

This invention is directed to methods and compositions for the treatment of cardiovascular disorders. Specifically, the invention is directed to compositions comprising vitamin E, statins and/or glutathione peroxidase mimetics; methods of treating diabetic patients expressing the Hp-2-2 haptoglobin genotype; a method of inhibiting or suppressing a cardiovascular disorder in a diabetic subject, treating cardiovascular disease in subjects exhibiting the haptoglobin Hp-2-2 genotype; and methods of treating cardiovascular disease in subjects exhibiting the haptoglobin Hp-2-2 genotype.

### BACKGROUND OF THE INVENTION

It is estimated that more than 13 million Americans are afflicted with clinically significant coronary artery disease (CAD) (American Heart Association 2004) and the care of these patients costs greater than $133 billion annually. Cardiovascular disease is the leading killer in America today. Over 50 million Americans have heart and cardiovascular related problems. By the time that cardiovascular heart problems are usually detected, the disease is usually quite advanced, having progressed for decades, and often too advanced to allow successful prevention of major permanent disability. Circulatory disease is caused by the normal flow of blood through the body being restricted or blocked as a result of arterial plaque. This may cause damage to the heart, brain, kidneys or other organs and tissues. Plaque build-up is a slow and progressive progress that is dependent on our environmental and genetic environment

A polymorphism in the haptoglobin (Hp) gene, an antioxidant protein, appears to permit identification of individuals with high oxidative stress and who may benefit from specially targeted therapy.

The identification of novel markers correlated with CVD is important in order to understand the pathophysiological mechanisms of these disorders state and develop targeted prevention and treatment regimens.

### SUMMARY OF THE INVENTION

In one embodiment, the invention provides a method of determining the potential of a diabetic subject having cardiovascular disorder to benefit from administration of a composition comprising vitamin E or its derivative, metabolite, or analog and their combination; and a statin; comprising the step of determining a haptoglobin phenotype of the subject, wherein a subject having a haptoglobin 2-2 phenotype will benefit from administration of the composition.

In another embodiment, the invention provides a method of determining the potential of a diabetic subject having cardiovascular disorder to benefit from administration of a composition comprising vitamin E or its derivative, metabolite, or analog and their combination; and a glutathione peroxidase (GPx) mimetic, or its isomer, functional derivative, synthetic analog, pharmaceutically acceptable salt or combination thereof; comprising the step of determining a haptoglobin phenotype of the subject, whereby a subject having a haptoglobin 2-2 phenotype will benefit from administration of the composition.

In another embodiment, the invention provides a method of determining the potential of a diabetic subject having cardiovascular disorder to benefit from administration of a composition comprising a statin and a GPx mimetic or its isomer, functional derivative, synthetic analog, pharmaceutically acceptable salt or combination thereof, comprising the step of determining a haptoglobin phenotype of the subject, whereby a subject having a haptoglobin 2-2 phenotype will benefit from administration of the composition.

In one embodiment, the invention provides a method of treating a cardiovascular disorder in a diabetic subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination, and a statin.

In another embodiment, the invention provides a method of inhibiting or suppressing a cardiovascular disorder in a diabetic subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a statin.

In one embodiment, the invention provides a method of alleviating symptoms associated with a cardiovascular disorder in a diabetic subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a statin.

In one embodiment, the invention provides a method of treating a cardiovascular disorder in a diabetic subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a GPx mimetic or its isomer, functional derivative, synthetic analog, pharmaceutically acceptable salt or combination thereof.

In another embodiment, the invention provides a method of inhibiting or suppressing a cardiovascular disorder in a diabetic subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a GPx mimetic or its isomer, functional derivative, synthetic analog, pharmaceutically acceptable salt or combination thereof.

In one embodiment, the invention provides a method of alleviating symptoms associated with a cardiovascular disorder in a diabetic subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a GPx mimetic or its isomer, functional derivative, synthetic analog, pharmaceutically acceptable salt or combination thereof.

In one embodiment, the invention provides a method of treating a cardiovascular disorder in a diabetic subject, comprising the steps of: obtaining a biological sample from the subject; determining the haptoglobin genotype of the subject; and if the subject's haptoglobin genotype is Hp-2-2, administering to the subject a composition comprising a statin; and a GPx mimetic or its isomer, functional derivative, synthetic analog, pharmaceutically acceptable salt or combination thereof.

In another embodiment, the invention provides a method of inhibiting or suppressing a cardiovascular disorder in a diabetic subject, comprising the steps of: obtaining a biological sample from the subject; determining the haptoglobin genotype of the subject; and if the subject's haptoglobin genotype is Hp-2-2, administering to the subject a composition comprising a statin; and a GPx mimetic or its isomer, functional derivative, synthetic analog, pharmaceutically acceptable salt or combination thereof.

In one embodiment, the invention provides a method of alleviating symptoms associated with a cardiovascular disorder in a diabetic subject, comprising the steps of: obtaining a biological sample from the subject; determining the haptoglobin genotype of the subject; and if the subject's haptoglobin genotype is Hp-2-2, administering to the subject a composition comprising a statin; and a GPx mimetic or its isomer, functional derivative, synthetic analog, pharmaceutically acceptable salt or combination thereof.

In another embodiment, the invention provides a method of determining the potential of a subject having cardiovascular disorder to benefit from administration of a composition comprising vitamin E or its derivative, metabolite, or analog and their combination; and a statin comprising the step of determining a haptoglobin phenotype of the subject, wherein a subject having a haptoglobin 2-2 phenotype will benefit from administration of the composition.

In one embodiment, the invention provides a method of determining the potential of a subject having cardiovascular disorder to benefit from administration of a composition comprising vitamin E or its derivative, metabolite, or analog and their combination; and a glutathione peroxidase (GPx) mimetic, or its isomer, functional derivative, synthetic analog, pharmaceutically acceptable salt or combination thereof, comprising the step of determining a haptoglobin phenotype of the subject, whereby a subject having a haptoglobin 2-2 phenotype will benefit from administration of the composition.

In another embodiment, the invention provides a method of determining the potential of a subject having cardiovascular disorder to benefit from administration of a composition comprising a statin and a GPx mimetic or its isomer, functional derivative, synthetic analog, pharmaceutically acceptable salt or combination thereof, comprising the step of determining a haptoglobin phenotype of the subject, whereby a subject having a haptoglobin 2-2 phenotype will benefit from administration of the composition.

In one embodiment, the invention provides a method of treating a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a statin.

In another embodiment, the invention provides a method of inhibiting or suppressing a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a statin.

In one embodiment, the invention provides a method of alleviating symptoms associated with a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a statin.

In one embodiment, the invention provides a method of treating a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a GPx mimetic or its isomer, functional derivative, synthetic analog, pharmaceutically acceptable salt or combination thereof.

In another embodiment, the invention provides a method of inhibiting or suppressing a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a GPx mimetic or its isomer, functional derivative, synthetic analog, pharmaceutically acceptable salt or combination thereof.

In one embodiment, the invention provides a method of alleviating symptoms associated with a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising a vitamin E, its analog, derivative or metabolite and their combination and a GPx mimetic or its isomer, functional derivative, synthetic analog, pharmaceutically acceptable salt or combination thereof.

In one embodiment, the invention provides a method of treating a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising a statin; and a GPx mimetic or its isomer, functional derivative, synthetic analog, pharmaceutically acceptable salt or combination thereof.

In another embodiment, the invention provides a method of inhibiting or suppressing a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising a statin; and a GPx mimetic or its isomer, functional derivative, synthetic analog, pharmaceutically acceptable salt or combination thereof.

In one embodiment, the invention provides a method of alleviating symptoms associated with a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising a statin; and a GPx mimetic or its isomer, functional derivative, synthetic analog, pharmaceutically acceptable salt or combination thereof.

In another embodiment, provided herein is a method of maintaining glycemic control in a diabetic subject comprising the step of bringing a diabetic subject exhibiting the Hp2-2 genotype's HbA_{1c} level down below 7.0% by administering to the subject a composition comprising a statin; and a vitamin E or its derivative, metabolite, or analog and/or their combination.

In another embodiment, provided herein is a method of maintaining glycemic control in a diabetic subject comprising the step of bringing a diabetic subject exhibiting the Hp2-2 genotype's HbA_{1c} level down below 7.0% by administering to the subject a composition comprising a statin; a vitamin E or its derivative, metabolite, or analog and/or their combination; and an agent or composition which lowers HbA_{1c} levels. In one embodiment, the agent is insuling or metformin.

In one embodiment, the invention provides a composition comprising: a statin; and a vitamin E or its derivative, metabolite, or analog and/or their combination. In one embodiment, the invention provides a pharmaceutical composition comprising: a statin; and a vitamin E or its derivative metabolite, or analog and/or their combination; and a diluent or carrier.

In another embodiment, the invention provides a composition comprising: a statin; and a glutathione peroxidase (GPx) mimetic; its isomer, functional derivative, synthetic analog, pharmaceutically acceptable salt or combination thereof. In another embodiment, the invention provides a composition comprising: a statin; and a glutathione peroxidase (GPx) mimetic; its isomer, functional derivative, synthetic analog, pharmaceutically acceptable salt or combination thereof; and a diluent or carrier.

In another embodiment, the invention provides a composition comprising: and a vitamin-E or its derivative metabolite, or analog and their combination; and a glutathione peroxidase (GPx) mimetic; its isomer, functional derivative, synthetic analog, pharmaceutically acceptable salt or combination thereof. In another embodiment, the invention provides a composition comprising: composition comprising: a vitamin-E or its derivative metabolite, or analog and their combination; and a glutathione peroxidase (GPx) mimetic; its isomer, functional derivative, synthetic analog, pharmaceutically acceptable salt or combination thereof, and a diluent or carrier.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWING

The invention will be better understood from a reading of the following detailed description taken in conjunction with the drawing, in which:

FIG. 1 shows Kaplan Meier plot of cardiovascular events in Hp 2-2 individuals receiving statins and randomized to placebo or vitamin E in the ICARE study.

### DETAILED DESCRIPTION OF THE INVENTION

This invention is directed to methods and compositions for the treatment of cardiovascular disorders. Specifically, the invention is directed to compositions comprising vitamin E, statins and/or glutathione peroxidase (GPx) mimetics; methods of treating diabetic patients expressing the Hp-2-2 haptoglobin genotype; a method of inhibiting or suppressing a cardiovascular disorder in a diabetic subject, treating cardiovascular disease in subjects exhibiting the Haptoglobin Hp-2-2 genotype; and methods of treating cardiovascular disease in subjects exhibiting the Haptoglobin Hp-2-2 genotype.

This invention relates in one embodiment to methods and compositions for the treatment of cardiovascular disorders. In another embodiment, provided herein are compositions comprising vitamin E, statins and/or glutathione peroxidase mimetics and methods of treating cardiovascular disease in subjects exhibiting the Haptoglobin Hp-2-2 genotype.

In one embodiment, the methods and compositions described herein, are effective in the treatment of diabetic patients, expressing the Hp-2-2 haptoglobin genotype. In another embodiment, the invention provides a method of inhibiting or suppressing a cardiovascular disorder in a diabetic subject, comprising the steps of: obtaining a biological sample from the subject; determining the haptoglobin genotype of the subject; and if the subject's haptoglobin genotype is Hp-2-2, administering to the subject a composition comprising vitamin E or its derivative, metabolite, or analog and their combination; and a statin, or a composition comprising vitamin E or its derivative, metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof.

Haptoglobin is inherited by two co-dominant autosomal alleles situated on chromosome 16 in humans, these are Hp1 and Hp2. There are three phenotypes Hp1-1, Hp2-1 and Hp2-2. The haptoglobin molecule is a tetramer comprising four polypeptide chains, two alpha and two beta chains, of which the alpha chain is responsible for polymorphism because it exists in two forms, alpha-1 and alpha-2. Hp1-1 is a combination of two alpha-1 chains along with two beta chains. Hp2-1 is a combination of one α-1 chain and one alpha-2 chain along with two beta chains. Hp2-2 is a combination of two α-2 chains and two beta chains. Hp1-1 individuals have greater hemoglobin binding capacity when compared to those individuals with Hp2-1 and Hp2-2. The gene differentiation to Hp-2 from Hp-1 resulted in a dramatic change in the biophysical and biochemical properties of the haptoglobin protein encoded by each of the 2 alleles. The haptoglobin phenotype of any individual, 1-1, 2-1 or 2-2, is readily determined, in one embodiment, from 10 μl of plasma by gel electrophoresis.

In one embodiment, antioxidant therapy may be beneficial in specific subgroups with increased oxidative stress. Oxidative stress refers in one embodiment to a loss of redox homeostasis (imbalance) with an excess of reactive oxidative species (ROS) by the singular process of oxidation. Both redox and oxidative stress are associated in another embodiment, with an impairment of antioxidant defensive capacity as well as an overproduction of ROS. In another embodiment, the methods and compositions of the invention are used in the treatment of complications or pathologies resulting from oxidative stress in subjects.

In one embodiment, activated neutrophils and tissue macrophages use an NADPH cytochrome b-dependent oxidase for the reduction of molecular oxygen to superoxide anions. In another embodiment, fibroblasts, are also be stimulated to produce ROS in response to pro-inflammatory cytokines. In another embodiment, prolonged production of high levels of ROS cause severe tissue damage. In one embodiment, high levels of ROS cause DNA mutations that can lead to neoplastic transformation. Therefore and in one embodiment, cells in injured tissues such as glial cells and neurons, must be able to protect themselves against the toxic effects of ROS. In one embodiment ROS-detoxifying enzymes have an important role in epithelial wound repair. In another embodiment, the glutathione peroxidase mimetics provided in the compositions and compounds provided herein, replace the ROS detoxifying enzymes described herein.

In one embodiment, overproduction of reactive oxygen species (ROS) including hydrogen peroxide (H₂O₂), superoxide anion (O⁻₂⁻); nitric oxide (NO⁻) and singlet oxygen (¹O₂) creates an oxidative stress, resulting in the amplification of the inflammatory response. Self-propagating lipid peroxidation (LPO) against membrane lipids begins and endothelial dysfunction ensues. Endogenous free radical scavenging enzymes (FRSEs) such as superoxide dismutase (SOD), glutathione peroxidase (GPx) and catalase are, involved in the disposal of O⁻₂⁻ and H₂O₂. First, SOD catalyses the dismutation of O⁻₂⁻ to H₂O₂ and molecular oxygen (O₂), resulting in selective O⁻₂⁻ scavenging. Then, GPx and catalase independently decompose H₂O₂ to H₂O. In another embodiment, ROS is released from the active neutrophils in the inflammatory tissue, attacking DNA and/or membrane lipids and causing chemical damage, including in one embodiment, to healthy tissue. When free radicals are generated in excess or when FRSEs are defective, H₂O₂ is reduced into hydroxyl radical (OH⁻), which is one of the highly reactive ROS responsible in one embodiment for initiation of lipid peroxidation of cellular membranes. In another embodiment, organic peroxide-induced lipid peroxidation is implicated as one of the essential mechanisms of toxicity in the death of hippocampal neurons. In one embodiment, an indicator of the oxidative stress in the cell is the level of lipid peroxidation and its final product is MDA. In another embodiment the level of lipid peroxidation increases in inflammatory diseases, such as meningitis in one embodiment. In one embodiment, some of the compounds provided herein and in another embodiment, are represented by the compounds of formula I and II, are effective antioxidants, capable of reducing lipid peroxidation, or in another embodiment, are effective as anti-inflammatory agents.

In one embodiment, cardiovascular disease refers to all disease which involves the heart and/or blood vessels, arteries, and occasionally veins. In one embodiment, the disease is a vascular disease. These problems are most commonly due to consequences of arterial disease, atherosclerosis, atheroma, but also can be related to infection, valvular and clotting problems.

In another embodiment, dual therapy with antioxidants and statins provides superior cardiovascular protection in a diabetic subject to Hp 2-2 individuals as compared to statins alone. In another embodiment, dual therapy with antioxidants and statins provides superior cardiovascular protection to Hp 2-2 individuals as compared to statins alone.

In one embodiment, the invention provides a composition comprising: a statin; and a vitamin E or its derivative, metabolite, or analog and their combination. In another embodiment, the invention provides a composition comprising: a statin; and a glutathione peroxidase (GPx) mimetic; its isomer, functional derivative, synthetic analog, pharmaceutically acceptable salt or combination thereof. In one embodiment, the invention provides a composition comprising: a glutathione peroxidase (GPx) mimetic; its isomer, functional derivative, synthetic analog, pharmaceutically acceptable salt or combination thereof; and a vitamin E or its derivative, metabolite, or analog and their combination.

In another embodiment, the term “statins” refers to a family of compounds that are inhibitors of 3-hydroxy-3-methylglutaryl coenzyme A (HMG-CoA) reductase, the rate-limiting enzyme in cholesterol biosynthesis. As HMG-CoA reductase inhibitors, in one embodiment, statins reduce plasma cholesterol levels in various mammalian species.

The statins used in the compositions and methods described herein, inhibit in one embodiment, cholesterol biosynthesis in humans by competitively inhibiting the 3-hydroxy-3-methyl-glutaryl-coenzyme A (“HMG-CoA”) reductase enzyme. HMG-CoA reductase catalyzes in another embodiment, the conversion of HMG to mevalonate, which is the rate determining step in the biosynthesis of cholesterol. Decreased production of cholesterol causes in one embodiment, an increase in the number of LDL receptors and corresponding reduction in the concentration of LDL particles in the bloodstream. Reduction in the LDL level in the bloodstream reduces the risk of coronary artery disease in one embodiment and other cardiovascular diseases in other embodiments.

Statins used in the compositions and methods of the invention are lovastatin (referred to as mevinolin in one embodiment, or monacolin-K in another embodiment), compactin (referred to as mevastatin in one embodiment, or ML-236B in another embodiment), pravastatin, atorvastatin (Lipitor), rosuvastatin (Crestor), fluvastatin (Lescol), simvastatin (Zocor), or cerivastatin. In one embodiment, the statin used as one or more additional therapeutic agent, is any one of the statins described herein, or in another embodiment, in combination of statins. A person skilled in the art would readily recognize that the choice of statin used, will depend on several factors, such as in certain embodiment, the underlying condition of the subject, other drugs administered, other pathologies and the like.

Vitamin E (alpha-tocopherol) is a fat soluble vitamin found in vegetable oils, egg yolk, milk fat, nuts, and cereal grains. Its primary functions are felt to be as a lipid antioxidant protecting lipids from oxidative modification.

In vitro data confirm the ability of vitamin E to prevent the oxidation of lipids. During incubation with cultured endothelial cells, the LDL particle undergoes various structural changes that will alter its metabolism. These changes are dependent on lipid peroxidation as an initial step. This oxidative modification can be totally inhibited by the addition of vitamin E to the cellular preparation. Vitamin E is a safe drug with few clinically important side effects. Animal studies have shown that vitamin E is not carcinogenic or teratogenic. In human studies few side effects have been reported in double-blind protocols and other large studies, even at high doses.

Natural vitamin E is available as a by-product of vegetable oil production, where it is extracted as the alcohol d-alpha-tocopherol, or as the synthesised acetate which is generally more stable than the alcohol. The natural (d-)forms are more active than the synthesised (dl-)form. Relative activities are: dl-alpha-tocopheryl acetate 1000 IU/g, dl-alpha-tocopherol 1100 IU/g, d-alpha-tocopheryl acetate 1360 IU/g, and d-alpha-tocopherol 1490 IU/g.

Vitamin E as used in the compositions and methods described herein refers to d-α-tocopherol, its derivative, metabolite, or analog and their combination. In one embodiment, derivatives or metabolites of vitamin E are used in the compositions and methods described herein and may comprise carboxyethylhydroxychromanes, or γ-tocopherol, tocotrienol, 2,7,8-triethyl-2-([beta]-carboxyethyl)-6-hydroxychromane, 2,5,7,8-tetramethyl-2-([beta]-carboxyethyl)-6-hydroxychromane, 2,7,8-trimethyl-2-([beta]-carboxyethyl)-6-hydroxychromane, in ceratin other embodiments. In one embodiment, “vitamin E” refers to all stereoisomeric forms of alpha-tocopherol, beta-tocopherol, gamma-tocopherol, delta-tocopherol, alpha-tocotrienol, beta-tocotrienol, gamma-tocotrienol and delta-tocotrienol together with the acetate and succinate esters of these compounds, or as chelated to any other organic acid. In one embodiment “vitamin E” is defined by the amount of IU—“International Units” rather than by chemical form, wherein one IU is equivalent to one mg dl-alpha-tocopheryl acetate.

In one embodiment, vitamin E is added to foods in one of its more chemically stable forms, e.g., alpha-tocopherol acetate (also known as alpha-tocopheryl acetate). Four different forms of vitamin E (the alcohol and ester forms of synthetic racemic (rac) vitamin E and the alcohol and ester forms of natural (RRR) vitamin E) are commercially available, and because of their differences in bioactivities and molecular weights, are assigned different values of specific activity (IU per milligram) according to the National Formulary as follows: 1 mg all-rac-.alpha.-tocopherol acetate=1.00 IU 1 mg all-rac-.alpha-tocopherol=1.10 IU 1 mg RRR-.alpha-tocopherol acetate=1.36 IU 1 mg RRR-.alpha-tocopherol=1.49 IU.

In one embodiment, the vitamin E is selected from the group consisting of alpha, beta, gamma and delta tocopherols, alpha, beta, gamma and delta tocotrienols, and combinations thereof. In another embodiment, the alpha tocopherol group is selected from the group consisting of synthetic (all-rac) and natural (RRR) alpha-tocopherols, alpha-tocopheryl acetates, and alpha-tocopheryl succinates.

Glutathione peroxidase (GPx) can be found largely in mammals cells, in mitochondrial matrix and cytoplasm. It reacts in one embodiment, with a large number of hydroperoxides (R—OOH). Glutathione peroxidase is of great importance within cellular mechanism for detoxification, since it is able in another embodiment, to reduces, in the same manner, the hydroperoxides from lipidic peroxidation. GPx is distributed extensively in cells, blood, and tissues, and its activity decreases when an organism suffers from diseases such as diabetes. In one embodiment, GPx is involved in many pathological conditions and is one of the most important antioxidant enzymes in living organisms.

However, the therapeutic usage of the native GPx is limited because of its instability, its limited availability, and the fact that is extremely difficult to prepare by using genetic engineering techniques because it contains selenocysteine encoded by the stop codon UGA.

Four types of GPx have been identified: cellular GPx (cGPx), gastrointestinal GPx, extracellular GPx, and phospholipid hydroperoxide GPx. cGPx, also termed in one embodiment, GPX1, is ubiquitously distributed. It reduces hydrogen peroxide as well as a wide range of organic peroxides derived from unsaturated fatty acids, nucleic acids, and other important biomolecules. At peroxide concentrations encountered under physiological conditions and in another embodiment, it is more active than catalase (which has a higher Kₘ for hydrogen peroxide) and is active against organic peroxides in another embodiment. Thus, cGPx represents a major cellular defense against toxic oxidant species.

Peroxides, including hydrogen peroxide (H₂O₂), are one of the main reactive oxygen species (ROS) leading to oxidative stress. H₂O₂ is continuously generated by several enzymes (including superoxide dismutase, glucose oxidase, and monoamine oxidase) and must be degraded to prevent oxidative damage. The cytotoxic effect of H₂O₂ is thought to be caused by hydroxyl radicals generated from iron-catalyzed reactions, causing subsequent damage to DNA, proteins, and membrane lipids.

In one embodiment, administration of a compound that mimics the biological activity of Gpx is achieved by administering a GPx mimetic or its pharmaceutically acceptable salt, its functional derivative, its synthetic analog or a combination thereof, which, in another embodiment, is a selenoorganic compound, is used in the methods and compositions of the invention. In another embodiment, the selenorganic compounds used in the methods and compositions of the invention, encompass selenoorganic compounds in which the selenium atom binds directly to a heteroatom such as nitrogen and generates the well-known GPx mimic, 2-phenyl-1,2-benziososelenazol-3(2H)-one (Ebselen™), or in another embodiment wherein the selenium atom is not directly bound to the heteroatom (N or O), but is instead located in close proximity to it; or in another embodiment, in which cyclodextrin is used as an enzyme model and the selenium is not directly bound or located in close proximity to the heteroatom. In one embodiment, slelnoorganic compounds prepared by any of the techniques described hereinabove are used in the compositions and methods of the invention. In one embodiment, the GPx mimetic used in the compositions and methods of this invention, is 4,4-dimethyl-benziso-2H-selenazine, also known as BXT-51072, ALT-2074 or SYI-2074.

In one embodiment, selenium and selenium-containing compounds are beneficial, exhibiting inter-alia anti-cancer properties, hepatoprotective properties and antiviral properties. The role of selenium in one embodiment, is to prevent free-radical damage either directly, through the incorporation into radical scavengers, or in another embodiment indirectly, through reduction of the byproducts of oxidative damage.

The selenorganic compounds used in the compositions and methods of the invention may in one embodiment be ebselen (2-phenyl-1,2-benzisoselenazol-3(2H)-one), used for hydroperoxide-inactivating therapy, with properties such as free radical and singlet oxygen quenching in one embodiment. In another embodiment, it can protect against oxidative challenge in vitro in liposomes, microsomes, isolated cells, and organs. In another embodiment, it has antiinflammatory properties and is effective for acute ischemic stroke without significant adverse effects.

In one embodiment, the effectiveness of the compounds provided herein derive from special structural features of the heterocyclic compounds provided herein. In one embodiment, having a large number of electrons in the π orbital overlap around the transition metal incorporated allows the formation of π-bonds and the donation of an electron to terminate free radicals formed by ROS. In one embodiment, the glutathione peroxidase mimetic used in the method of inhibiting or suppressing free radical formation, causing in another embodiment, lipid peroxidation and inflammation, is the product of formula (I):

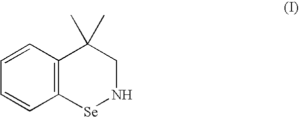

where nitrogen has 4 electrons in the p-orbital, thereby making 2 electrons available for π bonds; and each carbon has 2 electron in the p-orbital thereby making 1 electron available for π bonds; and selenium has 6 electrons in the p-orbital, thereby making 3 electrons available for π bonds, for a total of 7 electrons, since in another embodiment, the adjacent benzene ring removes two carbons from participating in the π-bond surrounding the metal. Upon a loss of electron by the transition metal, following termination of free radicals, the number of electrons in the π-bond overlap, is reduced to 6 π electron, a very stable aromatic sextet. In vitro and in vivo studies with the compound of formula I, a show in one embodiment, that glutathione peroxidase or its isomer, metabolite, and/or salt therefore is capable of protecting cells against reactive oxygen species.

In another embodiment, compositions and methods of the invention refer to benzisoselenazoline or -azine derivatives represented by the following general formula:

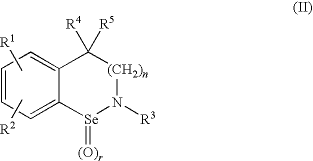

where
R¹, R²=hydrogen; lower alkyl; OR⁶; —(CH₂)ₘ NR⁶R⁷; —(CH₂)_{q}NH₂; —(CH₂)ₘ NHSO₂ (CH₂)₂ NH₂; —NO₂; —CN; —SO₃H; —N⁺ (R⁵)₂ O—; F; Cl; Br; I; —(CH₂)ₘ R⁸; —(CH₂)ₘ COR⁸; —S(O)NR⁶ R⁷; —SO₂ NR⁶ R⁷; —CO(CH₂)ₚ COR⁸; R⁹; R³=hydrogen; lower alkyl; aralkyl; substituted aralkyl; —(CH₂)ₘ COR⁸; —(CH₂)_{q}R⁸; —CO(CH₂)ₚ COR⁸; —(CH₂)ₘ SO₂ R⁸; —(CH₂)ₘ S(O)R⁸; R⁴=lower alkyl; aralkyl; substituted aralkyl; —(CH₂)ₚ COR⁸; —(CH₂)ₚR⁸; F; R⁵=lower alkyl; aralkyl; substituted aralkyl; R⁶ lower alkyl; aralkyl; substituted aralkyl; —(CH₂)ₘCOR⁸; —(CH₂)_{q}R⁸; R⁷ lower alkyl; aralkyl; substituted aralkyl; —(CH₂)ₘCOR⁸; R⁸ lower alkyl; aralkyl; substituted aralkyl; aryl; substituted aryl; heteroaryl; substituted heteroaryl; hydroxy; lower alkoxy; R⁹; R⁹=

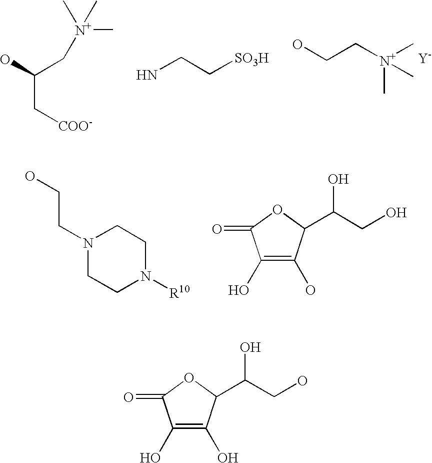

R¹⁰ hydrogen; lower alkyl; aralkyl or substituted aralkyl; aryl or substituted aryl; Y⁻ represents the anion of a pharmaceutically acceptable acid; n=0, 1; m=0, 1, 2; p=1, 2, 3; q=2, 3, 4 and r=0, 1.

In one embodiment, “Alkyl” refers to monovalent alkyl groups preferably having from 1 to about 12 carbon atoms, more preferably 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alky” refers to alkyl groups having 1 to 6 carbon atoms.

In another embodiment, “Aralkyl” refers to -alkylene-aryl groups preferably having from 1 to 10 carbon atoms in the alkylene moiety and from 6 to 14 carbon atoms in the aryl moiety. Such alkaryl groups are exemplified by benzyl, phenethyl, and the like.

“Aryl” refers in another embodiment, to an unsaturated aromatic carbocyclic group of from 6 to 14 carbon atoms having a single ring (e.g., phenyl) or multiple condensed rings (e.g., naphthyl or anthryl). Preferred aryls include phenyl, naphthyl and the like. Unless otherwise constrained by the definition for the individual substituent, such aryl groups can optionally be substituted with from 1 to 3 substituents selected from the group consisting of alkyl, substituted alkyl, alkoxy, alkenyl, alkynyl, amino, aminoacyl, aminocarbonyl, alkoxycarbonyl, aryl, carboxyl, cyano, halo, hydroxy, nitro, trihalomethyl and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —C(O)N(Rₓ)₂; —OC(O)Rₓ; —OCO₂Rₓ; —OC(O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety.

In one embodiment, the glutathione peroxidase mimetic or its isomer, metabolite, and/or salt therefore, used in the methods and compositions provided herein is an organoselenium compound. The term “organoselenium” refers in one embodiment to organic compound comprising at least one selenium atom. Preferred classes of organoselenium glutathione peroxidase mimetics include benzisoselenazolones, diaryl diselenides and diaryl selenides. In one embodiment, provided herein are compositions and methods of treating cardiovascular complication in a diabetic subject, comprising organoselenium compounds, thereby increasing endogenous anti-oxidant ability of the cells, or in another embodiment, scavenging free radicals causing apoptosis of cardiovascular organs and tissues and their associated pathologies.

Non-limiting examples of GPx mimetics are describe in U.S. patents

Biologically active derivatives or analogs of the proteins described herein include in one embodiment peptide mimetics. Peptide mimetics can be designed and produced by techniques known to those of skill in the art. (see e.g., U.S. Pat. Nos. 4,612,132; 5,643,873 and 5,654,276, the teachings of which are incorporated herein by reference). These mimetics can be based, for example, on the protein's specific amino acid sequence and maintain the relative position in space of the corresponding amino acid sequence. These peptide mimetics possess biological activity similar to the biological activity of the corresponding peptide compound, but possess a “biological advantage” over the corresponding amino acid sequence with respect to, in one embodiment, the following properties: solubility, stability and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Other forms of the proteins and polypeptides described herein and encompassed by the claimed invention, include in another embodiment, those which are “functionally equivalent.” In one embodiment, this term, refers to any nucleic acid sequence and its encoded amino acid which mimics the biological activity of the protein, or polypeptide or functional domains thereof in other embodiments.

In certain embodiments, a GPx mimetic refers to glutathione peroxidase as described above, which may be a purified or recombinantly expressed protein, or a partially synthetically prepared protein to comprise the selenocysteine moiety.

Accordingly and in another embodiment, the invention provides a method of determining the potential of a subject having cardiovascular disorder in a diabetic subject to benefit from administration of a composition comprising vitamin E or its derivative metabolite, or analog and their combination; and a statin, or a composition comprising vitamin E or its derivative metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof, comprising the step of determining a haptoglobin phenotype of the subject, whereby a subject having a haptoglobin 2-2 phenotype will benefit from administration of any one of the compositions described herein.

Accordingly and in another embodiment, the invention provides a method of determining the potential of a subject having cardiovascular disorder to benefit from administration of a composition comprising vitamin E or its derivative metabolite, or analog and their combination; and a statin, or a composition comprising vitamin E or its derivative metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof, comprising the step of determining a haptoglobin phenotype of the subject, whereby a subject having a haptoglobin 2-2 phenotype will benefit from administration of any one of the compositions described herein.

In another embodiment, the antioxidant function of Hp is due to its ability to neutralize hemoglobin which is capable of generating the highly reactive hydroxyl radical. Micro-hemorrhages resulting in liberation of extravascular extracorpuscular hemoglobin are of increased frequency and severity in diabetic atherosclerosis. The Hp 1-1 protein is superior to the Hp 2-2 protein in protecting against extracorpuscular hemoglobin as a result of its better ability to prevent release of heme from the Hp-hemoglobin complex and to promote uptake of the Hp-hemoglobin complex via the macrophage CD163 receptor.

In another embodiment, the methods and systems provided herein of determining the potential of a subject having cardiovascular disorder in a diabetic subject to benefit from administration or supplementation of a combination of a vitamin E, a statin, and a GPx mimetic, comprising the step of obtaining a biological sample from the subject; and determining the subject's haptoglobin allelic genotype, whereby a subject expressing the Hp-2-2 genotype will benefit from supplementation of vitamin E, is effected by a signal amplification method, whereby said signal amplification method is PCR, LCR (LAR), Self-Sustained Synthetic Reaction (3SR/NASBA), Q-Beta (Qβ) Replicase reaction, or a combination thereof.

In another embodiment, the methods and systems provided herein of determining the potential of a subject having cardiovascular disorder to benefit from administration or supplementation of a combination of a vitamin E, a statin and a Gpx mimetic comprising the step of obtaining a biological sample from the subject; and determining the subject's haptoglobin allelic genotype, whereby a subject expressing the Hp-2-2 genotype will benefit from supplementation of vitamin E, is effected by a signal amplification method, whereby said signal amplification method is PCR, LCR (LAR), Self-Sustained Synthetic Reaction (3SR/NASBA), Q-Beta (Qβ) Replicase reaction, or a combination thereof.

In another embodiment, the methods and systems provided herein of determining the potential of a subject having cardiovascular disorder to benefit from administration of a composition comprising vitamin E or its derivative metabolite, or analog and their combination; and a statin, or a composition comprising vitamin E or its derivative metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof, comprising the step of determining a haptoglobin phenotype of the subject, is effected by a signal amplification method, whereby said signal amplification method is PCR, LCR (LAR), Self-Sustained Synthetic Reaction (3SR/NASBA), Q-Beta (Qβ) Replicase reaction, or a combination thereof.

In another embodiment, the signal amplification methods provided herein, which in another embodiment, can be carried out using the systems provided herein, may amplify a DNA molecule or an RNA molecule. In another embodiment, signal amplification methods used as part of the present invention include, but are not limited to PCR, LCR (LAR), Self-Sustained Synthetic Reaction (3SR/NASBA) or a Q-Beta (Q.beta.) Replicase reaction.

Polymerase Chain Reaction (PCR): The polymerase chain reaction (PCR), refers in one embodiment to a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, in one embodiment, they are said to be “PCR-amplified.”

Ligase Chain Reaction (LCR or LAR): The ligase chain reaction [LCR; referred to, in another embodiment as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed in one embodiment and DNA ligase is added to the mixture. Provided that there is complete complementarity at the junction, ligase will covalently link each set of hybridized molecules. In another embodiment of LCR, two probes are ligated together only when they base-pair with sequences in the target sample, without gaps or mismatches. Repeated cycles of denaturation, and ligation amplify a short segment of DNA. LCR has is used in combination with PCR in one embodiment, to achieve enhanced detection of single-base changes. In another embodiment, because the four oligonucleotides used in this assay can pair to form two short ligatable fragments, there is the potential for the generation of target-independent background signal. The use of LCR for mutant screening is limited in another embodiment, to the examination of specific nucleic acid positions.

Self-Sustained Synthetic Reaction (3SR1NASBA): The self-sustained sequence replication reaction (3SR) refers in one embodiment, to a transcription-based in vitro amplification system that can exponentially amplify RNA sequences at a uniform temperature. The amplified RNA is utilized in certain embodiments, for mutation detection. In an embodiment of this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In one embodiment of the method, a probe which recognizes the sequence of interest is attached to the replicatable RNA template for Qβ. replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37° C.). This prevents the use of high temperature as a means of achieving specificity as in the LCR, the ligation event can be used to detect a mutation at the junction site, but not elsewhere.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction (Mullis, PCR Methods Applic., 1:1, 1991). If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

In another embodiment, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

In another embodiment, the methods and systems provided herein of determining the potential of a subject having cardiovascular disorder to benefit from administration of a composition comprising vitamin E or its derivative metabolite, or analog and their combination; and a statin, or a composition comprising vitamin E or its derivative metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof, comprising the step of determining a haptoglobin phenotype of the subject, is effected by a direct detection method such as a cycling probe reaction (CPR), or a branched DNA analysis, or a combination thereof in other embodiments.

The direct detection method according to one embodiment is a cycling probe reaction (CPR) or a branched DNA analysis. When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Traditional methods of direct detection including Northern and Southern band RNase protection assays usually require the use of radioactivity and are not amenable to automation. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

In another embodiment, the methods and systems provided herein of determining the potential of a subject having cardiovascular disorder to benefit from administration of a composition comprising vitamin E or its derivative metabolite, or analog and their combination; and a statin, or a composition comprising vitamin E or its derivative metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof, comprising the step of determining a haptoglobin phenotype of the subject, is effected by at least one sequence change, which employs in one embodiment a restriction fragment length polymorphism (RFLP analysis), or an allele specific oligonucleotide (ASO) analysis, a Denaturing/Temperature Gradient Gel Electrophoresis (DGGE/TGGE), a Single-Strand Conformation Polymorphism (SSCP) analysis or a Dideoxy fingerprinting (ddF) or their combination in other embodiments.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations (Eckstein and Lilley (eds.), Nucleic Acids and Molecular Biology, vol. 2, Springer-Verlag, Heidelberg, 1988). Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered (Barlow and Lehrach, Trends Genet., 3:167, 1987). Recently, endonucleases encoded by group I introns have been discovered that might have greater than 12 base-pair specificity (Perhnan and Butow, Science 246:1106, 1989), but again, these are few in number.

Allele specific oligonucleotide (ASO): allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes (Vogelstein et al., N. Eng. J. Med., 319:525-532, 1988; and Farr et al., Proc. Natl. Acad. Sci., 85:1629-1633, 1988), and gsp/gip oncogenes (Lyons et al., Science 249:655-659, 1990). Because of the presence of various nucleotide changes in multiple positions, the ASO method requires the use of many oligonucleotides to cover all possible oncogenic mutations.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

In another embodiment, the methods and systems provided herein of determining the potential of a subject having cardiovascular disorder to benefit from administration of a composition comprising vitamin E or its derivative, metabolite, or analog and their combination; and a statin, or a composition comprising vitamin E or its derivative, metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof, comprising the step of determining a haptoglobin phenotype of the subject, may be accomplished directly in one embodiment, by analyzing the protein gene products of the haptoglobin gene, or portions thereof. Such a direct analysis is often accomplished using an immunological detection method.

In one embodiment, the methods and systems provided herein of determining the potential of a subject having cardiovascular disorder to benefit from administration of a composition comprising vitamin E or its derivative, metabolite, or analog and their combination; and a statin, or a composition comprising vitamin E or its derivative, metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof, comprising the step of determining a haptoglobin phenotype of the subject, by an immunological detection method, such as is a radio-immunoassay (RIA) in one embodiment, or an enzyme linked immunosorbent assay (ELISA), a western blot, an immunohistochemical analysis, or fluorescence activated cell sorting (FACS), or a combination thereof in other embodiments.

Immunological detection methods are fully explained in, for example, “Using Antibodies: A Laboratory Manual” (Ed Harlow, David Lane eds., Cold Spring Harbor Laboratory Press (1999)) and those familiar with the art will be capable of implementing the various techniques summarized hereinbelow as part of the present invention. All of the immunological techniques require antibodies specific to at least one of the two haptoglobin alleles. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

Radio-immunoassay (RIA): In one version, this method involves precipitation of the desired substrate, haptoglobin in this case and in the methods detailed hereinbelow, with a specific antibody and radiolabelled antibody binding protein (e.g., protein A labeled with I.sup.125) immobilized on a precipitable carrier such as agarose beads. The number of counts in the precipitated pellet is proportional to the amount of substrate. In an alternate version of the RIA, A labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a calorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Sandwich ELISA measures the amount of antigen between two layers of antibodies (i.e. capture and detection antibody). The antigen to be measured must contain at least two antigenic sites capable of binding to antibody, since at least two antibodies act in the sandwich. Either monoclonal or polyclonal antibodies can be used as the capture and detection antibodies in Sandwich ELISA systems. Monoclonal antibodies recognise a single epitope that allows fine detection and quantification of small differences in antigen. A polyclonal is often used as the capture antibody to pull down as much of the antigen as possible. The advantage of Sandwich ELISA is that the sample does not have to be purified before analysis, and the assay can be very sensitive (up to 2 to 5 times more sensitive than direct or indirect).

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, calorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

It will be appreciated by one ordinarily skilled in the art that determining the haptoglobin phenotype of an individual, either directly or genetically, may be effected using any suitable biological sample derived from the examined individual, including, but not limited to, blood, plasma, blood cells, saliva or cells derived by mouth wash, and body secretions such as urine and tears, and from biopsies, etc.

In one embodiment, the invention provides a method for treating a cardiovascular disorder in a subject, comprising administering to said subject a therapeutically effective amount of the compositions of the invention, wherein the cardiovascular disorder is selected from a coronary artery disease, or an aneurysm, an arteriosclerosis, an atherosclerosis, a myocardial infarction, a myocardial fibrosis, an embolism, a stroke, a thrombosis, an angina, a vascular plaque inflammation, a vascular plaque rupture, a hypertension, an edema, a primary aldosteronism, a Kawasaki disease, an angiotensin II/N^{G}-Nitro-L-Arginine Methyl Ester-Induced Myocardial Injury, an Ischemia-reperfusion myocardial injury, restenosis after coronary angioplasty, restenosis after stent placement, a calcification and an inflammation in other embodiments.

In one embodiment, the term “therapeutically effective amount” refers to a prophylactic amount, an amount effective for preventing or protecting against cardiovascular diseases, related diseases, and symptoms thereof, and amounts effective for alleviating or healing cardiovascular diseases, related diseases, and symptoms thereof in another embodiment. By administering a composition of the methods of the invention concurrently with a therapeutic cardiovascular compound, the therapeutic cardiovascular compound may be administered in one embodiment, in a dosage amount that is less than the dosage amount required when the therapeutic cardiovascular compound is administered as a sole active ingredient. By administering lower dosage amounts of the active ingredient, the side effects associated in one embodiment, therewith are reduced.

In one embodiment, the invention provides a method of treating a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising vitamin E or its derivative, metabolite, or analog and their combination; and a statin, or a composition comprising vitamin E or its derivative, metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof.

In another embodiment, the invention provides a method of inhibiting or suppressing a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising vitamin E or its derivative, metabolite, or analog and their combination; and a statin, or a composition comprising vitamin E or its derivative, metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof.

In one embodiment, the invention provides a method of alleviating symptoms associated with a cardiovascular disorder in a subject, comprising the steps of: obtaining a biological sample from the subject; determining the haptoglobin genotype of the subject; and if the subject's haptoglobin genotype is Hp-2-2, administering to the subject a composition comprising vitamin E or its derivative, metabolite, or analog and their combination; and a statin, or a composition comprising vitamin E or its derivative, metabolite, or analog and their combination; and a GPx mimetic or its isomer, functional derivative, synthetic analog, pharmaceutically acceptable salt or combination thereof in another embodiment; or a composition comprising a statin and a GPx mimetic or its isomer, functional derivative, synthetic analog, pharmaceutically acceptable salt or combination thereof.

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combination thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

The term “preventing” refers in another embodiment, to preventing the onset of clinically evident pathologies associated with CVD altogether, or preventing the onset of a preclinically evident stage of pathologies associated with CVD in individuals at risk, which in one embodiment are subjects exhibiting the Hp-2 allele. In another embodiment, the determination of whether the subject carries the Hp-2 allele, or in one embodiment, which Hp allele, precedes the methods and the step of administration of the compositions of the invention.

In another embodiment, the route of administration in the step of contacting in the methods of the invention, using the compositions described herein, is optimized for particular treatments regimens. If chronic treatment of plaques is required, in one embodiment, administration will be via continuous subcutaneous infusion, using in another embodiment, an external infusion pump. In another embodiment, if acute treatment of plaque rupture is required, such as in one embodiment, in the case of interplaque hemorrhage, then intravenous infusion is used.

As mentioned above, haptoglobin (Hp) is a highly conserved plasma glycoprotein and is the major protein that binds free hemoglobin (Hb) with a high avidity (kd, ˜1×10⁻¹⁵ mol/L). Ischemia-reperfusion is associated with intravascular hemolysis and hemoglobin (Hb) release into the bloodstream. Extracorpuscular hemoglobin (Hb) is rapidly bound by Hp. The role of the Hp-Hb complex in modulating oxidative stress and inflammation after ischemia-reperfusion is Hp genotype dependent.

Hp in subjects with the Hp 1-1 phenotype is able to bind more hemoglobin on a Molar basis than Hps containing products of the haptoglobin 2 allele. Haptoglobin molecules in subjects with the haptoglobin 1-1 phenotype are also more efficient antioxidants, since the smaller size of haptoglobin 1-1 facilitates in one embodiment, its entry to extravascular sites of oxidative tissue injury compared to products of the haptoglobin 2 allele. In another embodiment, this also includes a significantly greater glomerular sieving of haptoglobin in subjects with Hp-1-1 phenotype.

In one embodiment haptoglobin 2-2 phenotype is used as an independent risk factor, in relation to target organ damage in refractory essential hypertension, or in relation to atherosclerosis (in the general population) and acute myocardial infarction or in relation to mortality from HIV infection in other embodiments. In another embodiment, haptoglobin 2-2 phenotype make subjects more prone to oxidative stress, therefore, haptoglobin 2-2 phenotype is used in one embodiment as a negative predictor for cardiovascular disease.

Microvascular disease is another vascular disease treated by the methods and compositions described herein, and may be characterized in one embodiment, by an unevenly distributed thickening (or hyalinization) of the intima of small arterioles, due in another embodiment, to the accumulation of type IV collagen in the basement membrane, or microaneurisyms of the arterioles, which compromises the extent of the maximal arteriolar dilation that can be achieved and impairs the delivery of nutrients and hormones to the tissues, or to remove waste in another embodiment. The vasculature distal to the arterioles may also be affected in one embodiment, such as by increased capillary basement membrane thickening, abnormalities in endothelial metabolism, or via impaired fibrinolysis, also resulting in reduced delivery of nutrients and hormones to the tissues, or waste removal in another embodiment. All these conditions may lead in certain embodiment to an overwhelming of the natural antioxidane enzyme system, as well as the resorption of lipids into the liver.

In one embodiment, complications arising out of microvascular disorders result in blood flow being disturbed by changes of the blood abnormalities (such as acceleration of platelet aggregation, increase of the blood viscosity and decrease of the red blood-cell deformity) or by changes of the blood vessel abnormalities (such as reduction of the production of nitric oxide from the endothelial cells of blood vessels and acceleration of the reactivity on vasoconstrictive substances), then the hypoxia of nerves is caused, and finally the nerves are degenerated.

The term “myocardial infarct” or “MI” refers in another embodiment, to any amount of myocardial necrosis caused by ischemia. In one embodiment, an individual who was formerly diagnosed as having severe, stable or unstable angina pectoris can be diagnosed as having had a small MI. In another embodiment, the term “myocardial infarct” refers to the death of a certain segment of the heart muscle (myocardium), which in one embodiment, is the result of a focal complete blockage in one of the main coronary arteries or a branch thereof. In one embodiment, subjects which were formerly diagnosed as having severe, stable or unstable angina pectoris, are treated according to the methods or in another embodiment with the compositions of the invention, upon determining these subjects carry the Hp-2 allele.

The term “ischemia-reperfusion injury” refers in one embodiment to a list of events including: reperfusion arrhythmias, microvascular damage, reversible myocardial mechanical dysfunction, and cell death (due to apoptosis or necrosis). These events may occur in another embodiment, together or separately. Oxidative stress, intracellular calcium overload, neutrophil activation, and excessive intracellular osmotic load explain in one embodiment, the pathogenesis and the functional consequences of the inflammatory injury in the ischemic-reperfused myocardium. In another embodiment, a close relationship exists between reactive oxygen species and the mucosal inflammatory process.

The term “stroke” is art recognized and is intended to include sudden diminution or loss of consciousness, sensation, and voluntary motion caused by rapture or abstraction (e.g. by a blood clot) of an artery of the brain.term. In another embodiment, “stroke” refers to the loss of oxygen supply to the brain, i.e., anoxia, with subsequent levels of glutamate and nitric oxide produced which are toxic to nerve cells. In another embodiment, stroke refers to the destruction of brain tissue due to impaired blood supply caused by intracerebral hemorrhage, thrombosis (clotting), or embolism (obstruction caused by clotted blood or other foreign matter circulating in the bloodstream). Stroke is a common cause of death in the United States. The method of treating a stroke with the compositions described herein, involves in another embodiment, administering to a subject a therapeutically effective amount of the compositions described herein such that universal distribution of the composition to the brain occurs, by introduction into the cerebrospinal fluid of the subject in certain embodiments.

In one embodiment haptoglobin protein impacts the development of atherosclerosis. The major function of serum haptoglobin is to bind free hemoglobin, which in another embodiment, is thought to help scavenge labile plasma iron (LPI) and prevent its loss in the urine and to serve as an antioxidant thereby protecting tissues against hemoglobin mediated tissue oxidation. The antioxidant capacity of the different haptoglobin differ in one embodiment, with the haptoglobin 1-1 protein appearing to confer superior antioxidant protection as compared to the other forms of the protein. Gross differences in size of the haptoglobin protein present in individuals with the different phenotypes explain in one embodiment, the apparent differences in the oxidative protection afforded by the different types of haptoglobin. Haptoglobin 1-1 is markedly smaller then haptoglobin 2-2 and thus more capable to sieve into the extravascular compartment and prevent in another embodiment, hemoglobin mediated tissue damage at sites of vascular injury. In one embodiment, the differences between the antioxidative efficiencies of the various Hp-phenotypes show the importance of determining the Hp phenotype being carried by the subject.

In one embodiment, the haptoglobin (Hp) genotype helps to identify patients with high levels of oxidative stress and who will benefit from targeted therapy with the compositions described herein. The Hp gene is polymorphic with two common classes of alleles denoted 1 and 2. It was demonstrated that the Hp 2 allele protein product is an inferior antioxidant compared to the Hp 1 allele protein product. The distribution of the three Hp genotypes in western societies is approximately 16% Hp 1-1, 36% Hp 2-2 and 48% Hp 2-1.

In another embodiment, the role of the Hp-Hb complex in modulating oxidative stress and inflammation after ischemia-reperfusion is Hp genotype dependent. In one embodiment, Hp 2-Hb complexes are associated with increased Labile Plasma Iron (LPI), particularly in the diabetic state, resulting in another embodiment, in increased iron-induced oxidative injury in Hp 2 allele-carrying subjects. In one embodiment, specific receptors for LPI exist on cardiomyocytes through which LPI mediates its toxic effects.

In another embodiment, the production of II-10 by the Hp-Hb complex is Hp genotype dependent with markedly greater II-10 production in Hp 1 mice after ischemia-reperfusion. II-10 is an anti-inflammatory cytokine which inhibits NF-κB activation, oxidative stress and polymorphonuclear cell infiltration after ischemia-reperfusion.

In one embodiment, interleukin 10 markedly attenuates ischemia-reperfusion injury by inhibiting NF-κB activation, or decreases oxidative stress and prevents polymorphonuclear cell infiltration in other embodiments. In another embodiment, Hp-Hb complex is formed early in the setting of an acute myocardial infarction secondary to hemolysis as evidenced by an acute fall in serum Hp levels. Hp 1-1-Hb complex induces in one embodiment, a marked increase in II-10 release from macrophages in vitro acting via the CD163 receptor. In one embodiment, a Hp genotype dependent differences in II-10 release exist in the PMBC's of a subject following non-lethal MI. In another embodiment, plasma levels of II-10 in Hp 2 carrying subjects after ischemia-reperfusion is not statistically significant from plasma levels of II-10 in Hp 2 carrying subjects prior to ischemia-reperfusion.

In one embodiment, the methods provided herein, using the compositions provided herein, further comprise contacting the subject with one or more additional agent, which is not a statin, a vitamin E or its derivative, metabolite, or a GPx mimetic or its isomer, functional derivative, synthetic analog, pharmaceutically acceptable salt or combination thereof. In another embodiment, the additional agent is an angiotensin-converting enzyme. In another embodiment, the additional agent is an angiotensin receptor AT₁ blocker (ARB). In another embodiment, the additional agent is an angiotensin II receptor antagonist. In another embodiment, the additional agent is a calcium channel blocker. In another embodiment, the additional agent is a diuretic. In another embodiment, the additional agent is digitalis. In another embodiment, the additional agent is a beta blocker. In another embodiment, the additional agent is a cholestyramine or in another embodiment, the additional agent is a combination thereof.

In one embodiment, the additional agent may be an anti-dyslipidemic agent such as (i) bile acid sequestrants such as, cholestyramine, colesevelem, colestipol, dialkylaminoalkyl derivatives of a cross-linked dextran; Colestid™; LoCholest™; and Questran™, and the like; (ii) HMG-CoA reductase inhibitors such as atorvastatin, itavastatin, fluvastatin, lovastatin, pravastatin, rivastatin, rosuvastatin, simvastatin, and ZD-4522, and the like; (iii) HMG-CoA synthase inhibitors; (iv) cholesterol absorption inhibitors such as stanol esters, beta-sitosterol, sterol glycosides such as tiqueside; and azetidinones such as ezetimibe, vytorin, and the like; (v) acyl coenzyme A-cholesterol acyl transferase (ACAT) inhibitors such as avasimibe, eflucimibe, KY505, SMP 797, and the like; (vi) CETP inhibitors such as JTT 705, torcetrapib, CP 532,632, BAY63-2149, SC 591, SC 795, and the like; (vii) squalene synthetase inhibitors; (viii) anti-oxidants such as probucol, and the like; (ix) PPAR.alpha. agonists such as beclofibrate, benzafibrate, ciprofibrate, clofibrate, etofibrate, fenofibrate, gemcabene, and gemfibrozil, GW 7647, BM 170744, LY518674; and other fibric acid derivatives, such as Atromid™, Lopid™ and Tricorm, and the like; (x) FXR receptor modulators such as GW 4064, SR 103912, and the like; (xi) LXR receptor such as GW 3965, T9013137, and XTCO179628, and the like; (xii) lipoprotein synthesis inhibitors such as niacin; (xiii) rennin angiotensin system inhibitors; (xiv) PPAR o partial agonists; (xv) bile acid reabsorption inhibitors, such as BARI 1453, SC435, PHA384640, S892.1, AZD7706, and the like; (xvi) PPAR.delta. agonists such as GW 501516, and GW 590735, and the like; (xvii) triglyceride synthesis inhibitors; (xviii) microsomal triglyceride transport (MTTP) inhibitors, such as inplitapide, LAB687, and CP346086, and the like; (xix) transcription modulators; (xx) squalene epoxidase inhibitors; (xxi) low density lipoprotein (LDL) receptor inducers; (xxii) platelet aggregation inhibitors; (xxiii) 5-LO or FLAP inhibitors; and (xiv) niacin receptor agonists.

In one embodiment, the additional agent administered as part of the compositions, used in the methods provided herein, is an anti-platelet agents (or platelet inhibitory agents). The term anti-platelet agents (or platelet inhibitory agents), refers in one embodiment to agents that inhibit platelet function by inhibiting the aggregation, or by adhesion or granular secretion of platelets in other embodiments. In another embodiment, the anti-platelet agents used in the compositions described herein include, but are not limited to, the various known non-steroidal anti-inflammatory drugs (NSAIDs) such as aspirin, ibuprofen, naproxen, sulindac, indomethacin, mefenamate, droxicam, diclofenac, sulfinpyrazone, piroxicam, and pharmaceutically acceptable salts or prodrugs thereof. In another embodiment, the anti-platelet agent is IIb/IIIa antagonists (e.g., tirofiban, eptifibatide, and abciximab), thromboxane-A2-receptor antagonists (e.g., ifetroban), thromboxane-A2-synthetase inhibitors, PDE-III inhibitors (e.g., dipyridamole), and pharmaceutically acceptable salts or prodrugs thereof. In another embodiment, the term anti-platelet agents (or platelet inhibitory agents), refers to ADP (adenosine diphosphate) receptor antagonists, which is in one embodiment, an antagonists of the purinergic receptors P₂Y₁ and P₂Y₁₂. In one embodiment, P₂Y₁₂ receptor antagonists is ticlopidine, clopidogrel, or their combination and pharmaceutically acceptable salts or prodrugs thereof.

In another embodiment, the additional agent administered as part of the compositions, used in the methods provided herein, is an anti-hypertensive agents such as (i) diuretics, such as thiazides, including chlorthalidone, chlorothiazide, dichlorphenamide, hydroflumethiazide, indapamide, and hydrochlorothiazide; loop diuretics, such as bumetamide, ethacrynic acid, furosemide, and torsemide; potassium sparing agents, such as amiloride, and triamteren; and aldosterone antagonists, such as spironolactone, epirenone, and the like; (ii) beta-adrenergic blockers such as acebutolol, atenolol, betaxolol, bevantolol, bisoprolol, bopindolol, carteolol, carvedilol, celiprolol, esmolol, indenolol, metoprolol, nadolol, nebivolol, penbutolol, pindolol, propranolol, sotalol, tertatolol, tilisolol, and timolol, and the like; (iii) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (iv) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (v) neutral endopeptidase inhibitors such as omapatrilat, cadoxatril and ecadotril, fosidotril, sampatrilat, AVE7688, ER4030, and the like; (vi) endothelin antagonists such as tezosentan, A308165, and YM62899, and the like; (vii) vasodilators such as hydralazine, clonidine, minoxidil, and nicotinyl alcohol, and the like; (viii) angiotensin II receptor antagonists such as candesartan, eprosartan, irbesartan, losartan, pratosartan, tasosartan, telmisartan, valsartan, and EXP-3137, FI6828K, and RNH6270, and the like; (ix) α/β adrenergic blockers as nipradilol, arotinolol and amosulalol, and the like; (x) alpha 1 blockers, such as terazosin, urapidil, prazosin, bunazosine, trimazosin, doxazocin, naftopidil, indoramin, WHIP 164, and XEN010, and the like; and (xi)-alpha 2 agonists such as lofexidine, tiamenidine, moxonidine, rilmenidine and guanabenz, and the like. Combinations of anti-obesity agents and diuretics or beta blockers may further include vasodilators, which widen blood vessels. Representative vasodilators useful in the compositions and methods of the present invention include, but are not limited to, hydralazine (apresoline), clonidine (catapres), minoxidil (loniten), and nicotinyl alcohol (roniacol).

The rennin-angiotensin-aldosterone system (“RAAS”) is involved in one embodiment, in regulating pressure homeostasis and also in the development of hypertension, a condition shown as a major factor in the progression of cardiovascular diseases. Secretion of the enzyme rennin from the juxtaglomerular cells in the kidney activates in another embodiment, the rennin-angiotensin-aldosterone system (RAAS), acting on a naturally-occurring substrate, angiotensinogen, to release in another embodiment, a decapeptide, Angiotensin I. Angiotensin converting enzyme (“ACE”) cleaves in one embodiment, the secreted decapeptide, producing an octapeptide, Angiotensin II, which is in another embodiment, the primary active species of the RAAS system. Angiotensin II stimulates in one embodiment, aldosterone secretion, promoting sodium and fluid retention, inhibiting rennin secretion, increasing sympathetic nervous system activity, stimulating vasopressin secretion, causing a positive cardiac inotropic effect or modulating other hormonal systems in other embodiments.

A representative group of ACE inhibitors consists in another embodiment, of the following compounds: AB-103, ancovenin, benazeprilat, BRL-36378, BW-A575C, CGS-13928C, CL-242817, CV-5975, Equaten, EU-4865, EU-4867, EU-5476, foroxymithine, FPL 66564, FR-900456, Hoe-065, I5B2, indolapril, ketomethylureas, KRI-1177, KRI-1230, L-681176, libenzapril, MCD, MDL-27088, MDL-27467A, moveltipril, MS-41, nicotianamine, pentopril, phenacetin, pivopril, rentiapril, RG-5975, RG-6134, RG-6207, RGH-0399, ROO-911, RS-10085-197, RS-2039, RS 5139, RS 86127, RU-44403, S-8308, SA-291, spiraprilat, SQ-26900, SQ-28084, SQ-28370, SQ-23940, SQ-31440, Synecor, utibapril, WF-10129, Wy-44221, Wy-44655, Y-23785, Yissum P-0154, zabicipril, Asahi Brewery AB-47, alatriopril, BMS 182657, Asahi Chemical C-111, Asahi Chemical C-112, Dainippon DU-1777, mixanpril, Prentyl, zofenoprilat, 1-(-(1-carboxy-6-(4-piperidinyl)hexyl)amino)-1-oxopropyl octahydro-1H-indole-2-carboxylic acid, Bioproject BP1.137, Chiesi CHF 1514, Fisons FPL-6564, idrapril, Marion Merrell Dow MDL-100240, perindoprilat and Servier S-5590, alacepril, benazepril, captopril, cilazapril, delapril, enalapril, enelaprilat, fosinopril, fosinoprilat, imidapril, lisinopril, perindopril, quinapril, ramipril, saralasin acetate, temocapril, trandolapril, ceranapril, moexipril, quinaprilat and spirapril.

In one embodiment, the terms “aldosterone antagonist” and “aldosterone receptor antagonist” refer to a compound that inhibits the binding of aldosterone to mineralocorticoid receptors, thereby blocking the biological effects of aldosterone. In one embodiment, the term “antagonist” in the context of describing compounds according to the invention refers to a compound that directly or in another embodiment, indirectly inhibits, or in another embodiment suppresses Aldosterone activity, function, ligand mediated transcriptional activation, or in another embodiment, signal transduction through the receptor. In one embodiment, antagonists include partial antagonists and in another embodiment full antagonists. In one embodiment, the term “full antagonist” refers to a compound that evokes the maximal inhibitory response from the Aldosterone, even when there are spare (unbound) Aldosterone present. In another embodiment, the term “partial antagonist” refers to a compound does not evoke the maximal inhibitory response from the androgen receptor, even when present at concentrations sufficient to saturate the androgen receptors present.

The aldosterone antagonists used in the methods and compositions of the present invention are in one embodiment, spirolactone-type steroidal compounds. In another embodiment, the term “spirolactone-type” refers to a structure comprising a lactone moiety attached to a steroid nucleus, such as, in one embodiment, at the steroid “D” ring, through a spiro bond configuration. A subclass of spirolactone-type aldosterone antagonist compounds consists in another embodiment, of epoxy-steroidal aldosterone antagonist compounds such as eplernone. In one embodiment, spirolactone-type antagonist compounds consists of non-epoxy-steroidal aldosterone antagonist compounds such as spironolactone. In one embodiment, the invention provides a composition comprising an aldosterone antagonist, its isomer, functional derivative, synthetic analog, pharmaceutically acceptable salt or combination thereof; and a glutathione peroxidase or its isomer, functional derivative, synthetic analog, pharmaceutically acceptable salt or combination thereof, wherein the aldosterone antagonist is epoxymexrenone, or eplernone, dihydrospirorenone, 2,2; 6,6-diethylene-3oxo-17alpha-pregn-4-ene-21,17-carbolactone, spironolactone, 18-deoxy aldosterone, 1,2-dehydro-18-deoxyaldosterone, RU28318 or a combination thereof in other embodiments.

In one embodiment, Cyclic fluxes of Ca²⁺ between three compartments—cytoplasm, sarcoplasmic reticulum (SR), and sarcomere—account for excitation-contraction coupling. Depolarization triggers in another embodiment, entry of small amounts of Ca²⁺ through the L-type Ca²⁺ channels located on the cell membrane, which in one embodiment, prompts SR Ca²⁺ release by cardiac ryanodine receptors (RyR's), a process termed calcium-induced Ca²⁺ release. A rapid rise in cytosolic levels results in one embodiment, fostering Ca²⁺-troponin-C interactions and triggering sarcomere contraction. In another embodiment, activation of the ATP-dependent calcium pump (SERCA) recycles cytosolic Ca²⁺ into the SR to restore sarcomere relaxation. In another embodiment, Ca²⁺ channel blockers inhibits the triggering of sarcomer contraction and modulate increase in systolic pressure.

In one embodiment, calcium channel blockers, are amlodipine, aranidipine, barnidipine, benidipine, cilnidipine, clentiazem, diltiazem, efonidipine, fantofarone, felodipine, isradipine, lacidipine, lercanidipine, manidipine, mibefradil, nicardipine, nifedipine, nilvadipine, nisoldipine, nitrendipine, semotiadil, verapamil, and the like. Suitable calcium channel blockers are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry, which can be used in the compositions and methods of the invention.

In another embodiment, the β-blocker used in the compositions and methods of the invention is propanalol, terbutalol, labetalol propranolol, acebutolol, atenolol, nadolol, bisoprolol, metoprolol, pindolol, oxprenolol, betaxolol or a combination thereof.

In one embodiment, a diuretic is used in the methods and compositions of the invention. In another embodiment, the diuretic is chlorothiazide, hydrochlorothiazide, methylclothiazide, chlorothalidon, or a combination thereof.

In one embodiment, the additional agent used in the compositions provided herein is a non-steroidal anti-inflammatory drug (NSAID). In another embodiment, the NSAID is sodium cromoglicate, nedocromil sodium, PDE4 inhibitors, leukotriene antagonists, iNOS inhibitors, tryptase and elastase inhibitors, beta-2 integrin antagonists and adenosine 2a agonists. In one embodiment, the NSAID is ibuprofen; flurbiprofen, salicylic acid, aspirin, methyl salicylate, diflunisal, salsalate, olsalazine, sulfasalazine, indomethacin, sulindac, etodolac, tolmetin, ketorolac, diclofenac, naproxen, fenoprofen, ketoprofen, oxaprozin, piroxicam, celecoxib, and rofecoxiband a pharmaceutically acceptable salt thereof. In one embodiment, the NSAID component inhibits the cyclo-oxygenase enzyme, which has two (2) isoforms, referred to as COX-1 and COX-2. Both types of NSAID components, that is both non-selective COX inhibitors and selective COX-2 inhibitors are useful in accordance with the present invention.

In another embodiment, the additional agent administered as part of the compositions, used in the methods provided herein, is a glycation inhibitor, such as pimagedine hydrochloride in one embodiment, or ALT-711, EXO-226, KGR-1380, aminoguanidine, ALT946, pyratoxanthine, N-phenacylthiazolium bromide (ALT766), pyrrolidinedithiocarbamate or their combination in yet another embodiment.

In one embodiment, the invention provides a composition comprising: a statin; and a vitamin E or its derivative, metabolite, or analog and/or their combination. In one embodiment, the invention provides a pharmaceutical composition comprising: a statin; and a vitamin E or its derivative, metabolite, or analog and/or their combination; and a diluent or carrier. In another embodiment, the invention provides a composition comprising: a statin; and a glutathione peroxidase (GPx) mimetic; its isomer, functional derivative, synthetic analog, pharmaceutically acceptable salt or combination thereof. In one embodiment, the invention provides a composition comprising: composition comprising: a statin; and a vitamin E or its derivative, metabolite, or analog and their combination; and a vitamin E or its derivative, metabolite, or analog and their combination.

In one embodiment, the composition further comprises a carrier, excipient, lubricant, flow aid, processing aid or diluent, wherein said carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetener, a film forming agent, or any combination thereof.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, or intracranially.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraarterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

The compounds utilized in the methods and compositions of the present invention may be present in the form of free bases in one embodiment or pharmaceutically acceptable acid addition salts thereof in another embodiment. In one embodiment, the term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I are prepared in another embodiment, from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, in another embodiment, the appropriate acid or base with the compound.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations of the invention can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

In one embodiment, the term “contacting” refers to bringing a subject in contact with the compositions provided herein. For example, in one embodiment, the compositions provided herein are suitable for oral administration, whereby bringing the subject in contact with the composition comprises ingesting the compositions. A person skilled in the art would readily recognize that the methods of bringing the subject in contact with the compositions provided herein, will depend on many variables such as, without any intention to limit the modes of administration; the cardiovascular disorder treated, age, pre-existing conditions, other agents administered to the subject, the severity of symptoms, location of the affected area and the like. In one embodiment, provided herein are embodiments of methods for administering the compounds of the present invention to a subject, through any appropriate route, as will be appreciated by one skilled in the art.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, the compositions of the invention are administered in conjunction with other therapeutica agents. Representative agents that can be used in combination with the compositions of the invention are agents used to treat diabetes such as insulin and insulin analogs (e.g. LysPro insulin); GLP-1 (7-37) (insulinotropin) and GLP-1 (7-36)—NH.sub.2; biguanides: metformin, phenformin, buformin; .alpha.2-antagonists and imidazolines: midaglizole, isaglidole, deriglidole, idazoxan, efaroxan, fluparoxan; sulfonylureas and analogs: chlorpropamide, glibenclamide, tolbutamide, tolazamide, acetohexamide, glipizide, glimepiride, repaglinide, meglitinide; other insulin secretagogues: linogliride, A-4166; glitazones: ciglitazone, pioglitazone, englitazone, troglitazone, darglitazone, rosiglitazone; PPAR-gamma agonists; fatty acid oxidation inhibitors: clomoxir, etomoxir; alpha.-glucosidase inhibitors: acarbose, miglitol, emiglitate, voglibose, MDL-25,637, camiglibose, MDL-73,945; beta.-agonists: BRL 35135, BRL 37344, Ro 16-8714, ICI D7114, CL 316,243; phosphodiesterase inhibitors: L-386,398; lipid-lowering agents: benfluorex; antiobesity agents: fenfluramine; vanadate and vanadium complexes (e.g. Naglivan.RTM.)) and peroxovanadium complexes; amylin antagonists; glucagon antagonists; gluconeogenesis inhibitors; somatostatin analogs and antagonists; antilipolytic agents: nicotinic acid, acipimox, WAG 994. Also contemplated for use in combination with the compositions of the invention are pramlintide acetate (Symlin.TM.), AC2993, glycogen phosphorylase inhibitor and nateglinide. Any combination of agents can be administered as described hereinabove.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

The term “subject” refers in one embodiment to a mammal including a human in need of therapy for, or susceptible to, a condition or its sequalae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Research Design and Methods

The study protocol of the ICARE study involves the following: participants were drawn from 47 primary health clinics of the Clalit Health Services in the northern sector of Israel. Patients were eligible for the study if they had Type II DM and were 55 years of age or older. 3054 individuals underwent Hp genotyping and of these 1434 were found to have the Hp 2-2 genotype. These Hp 2-2 individuals were randomly assigned to treatment with either vitamin E or placebo. The major study outcomes (MI, stroke, CVD death) were identified prospectively in this population over an 18 month period. A preplanned secondary analysis of ICARE was to assess the ability of vitamin E therapy to influence outcomes in those ICARE participants who were also taking statins. Statin use as prospectively defined in ICARE was based on the use of statins by the participant in at least eight of the twelve months preceding enrollment of the participant in the study. The decision to use statins for a particular participant was under the discretion of the patient's primary care physician and was in no way influenced by the patient's participation in the ICARE study

### Example 1

### Dual Therapy with Statins and Antioxidants is Superior to Statins Alone in Decreasing the Risk of Cardiovascular Disease in Individuals with Diabetes Mellitus and the Haptoglobin 2-2 Genotype

### Background

Robust clinical data has shown that individuals homozygous for the Hp 2 allele (Hp 2-2 genotype), 40% of DM individuals, have an up to 500% increased risk of CVD. A vast amount of basic science, animal and epidemiological data has provided the logic for targeting vitamin E administration specifically to DM individuals with the Hp 2-2 genotype.

In the ICARE study (Israel CArdiovascular events Reduction with vitamin E (ClinicalTrials.gov# NCT00220831)) a prospective randomized placebo controlled trial of vitamin E therapy in DM individuals with the Hp 2-2 genotype, is was shown that vitamin E therapy results in a 50% reduction in CVD events. However, only about half of the Hp 2-2 DM participants in ICARE received statin therapy. Because statin therapy is currently recommended for all DM individuals we sought to determine if antioxidant therapy could still be demonstrated to provide benefit to Hp 2-2 DM individuals also taking statins in ICARE

### Results

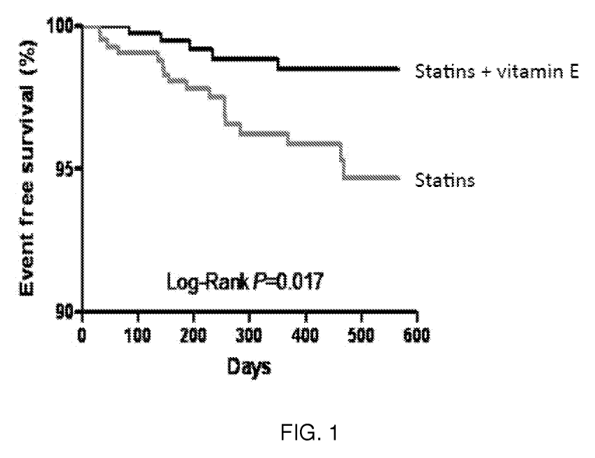

Of the 801 Hp 2-2 individuals taking statins in the ICARE cohort, 386 were randomized to vitamin E and 415 to placebo. There was no significant difference in the baseline characteristics, concurrent medications, or diabetes characteristics between those individuals taking statins who were randomized to placebo or vitamin E. It was found that dual treatment with statins and vitamin E dramatically reduced the event rate compared to statin treatment alone. (1.3% (5/386) for vitamin E vs. 4.1% (17/415) for placebo, hazard ratio [HR] 0.31, 95% confidence interval [CI] 0.15-0.83, p=0.017 by log-rank FIG. 1).

This magnitude of the beneficial affect of vitamins in those taking statins was unchanged if the definition for statin use was widened to include those patients taking statins in at least one of the twelve months preceding enrollment of the participant in the study (1.7% (9/538) for vitamin E vs. 4.2% (23/543) for placebo, p=0.013).

Having described embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1.-54. (canceled)

55. A method of determining the potential of a subject having a cardiovascular disorder to benefit from administration of vitamin E or its derivative, metabolite, analog or combination thereof; and a statin, comprising the step of determining a haptoglobin phenotype of the subject, wherein a subject having a haptoglobin 2-2 phenotype will benefit from administration thereof.

56. The method of claim 55 wherein the subject is diabetic.

57. The method of claim 55 wherein the vitamin E is natural vitamin E, d-δ-tocopherol, mixed tocopherol concentrate, its derivative, metabolite, analog, or combination thereof.

58. The method of claim 55 wherein the statin is lovastatin, compactin, pravastatin, atorvastatin, itavastatin, rosuvastatin, rivastatin, fluvastatin, simvastatin, cerivastatin, or combination thereof.

59. The method of claim 55 wherein the cardiovascular disorder is myocardial infarct, cardiovascular death, stroke, or a combination thereof.

60. A method of treating, inhibiting or suppressing a cardiovascular disorder or alleviating a symptom associated therewith in a subject, comprising the steps of:
a. obtaining a biological sample from the subject;
b. determining the haptoglobin genotype of the subject; and
c. if the subject's haptoglobin genotype is Hp 2-2, administering to the subject a vitamin E, its analog, derivative, metabolite or combination thereof; and a statin.

61. The method of claim 60 wherein the subject is diabetic.

62. The method of claim 60 wherein the vitamin E is natural vitamin E, d-δ-tocopherol, mixed tocopherol concentrate, its derivative, metabolite, analog, or a combination thereof.

63. The method of claim 60 wherein the statin is lovastatin, compactin, pravastatin, atorvastatin, itavastatin, rosuvastatin, rivastatin, fluvastatin, simvastatin, cerivastatin, or combination thereof.

64. The method of claim 60 wherein the cardiovascular disorder is myocardial infarct, cardiovascular death, stroke, or a combination thereof.

65. The method of claim 60 wherein the biological sample is blood, plasma, blood cells, saliva, cells derived by mouth wash, urine tears, biopsies, semen or a combination thereof.

66. A composition comprising:
a. a statin; and
b. vitamin E or its derivative, metabolite, analog, or combination thereof.

67. The composition of claim 66 wherein the statin is lovastatin, compactin, pravastatin, atorvastatin, itavastatin, rosuvastatin, rivastatin, fluvastatin, simvastatin, cerivastatin, or combination thereof.

68. The composition of claim 66 wherein the vitamin E is natural vitamin E, d-δ-tocopherol, mixed tocopherol concentrate, its derivative, metabolite, analog or combination thereof.

69. The method of claim 55 whereby said step of determining said haptoglobin genotype is effected by a method selected from a signal amplification method, a direct detection method, detection of at least one sequence change, immunological method or a combination thereof.

70. The method of claim 69, whereby said signal amplification method amplifies a molecule selected from the group consisting of a DNA molecule and an RNA molecule.

71. The method of claim 69, whereby said signal amplification method is selected from the group consisting of PCR, LCR (LAR), Self-Sustained Synthetic Reaction (3SR/NASBA) and Q-Beta (Qβ) Replicase reaction.

72. The method of claim 69, whereby said direct detection method is selected from the group consisting of a cycling probe reaction (CPR) and a branched DNA analysis.

73. The method of claim 69, whereby said detection of at least one sequence change employs a method selected from the group consisting of restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis and Dideoxy fingerprinting (ddF).

74. The method of claim 69, whereby step of determining said haptoglobin genotype is effected by an immunological detection method.

75. The method of claim 74, whereby said immunological detection method is a radio-immunoassay (RIA), an enzyme linked immunosorbent assay (ELISA), a Sandwich ELISA, a western blot, an immunohistochemical analysis, or fluorescence activated cell sorting (FACS).
